(19) United States
(12) Patent Application Publication
(10) Pub. No.: US 2011/0282135 A1
(43) Pub. Date: Nov. 17, 2011

(54) Device and method for artificial insemination of bovines

(76) Inventor: Todd Richard Waybright, State College, PA (US)

(21) Appl. No.: 12/662,979

(22) Filed: May 14, 2010

(51) Int. Cl. A61B 17/43

(52) U.S. Cl. 600/35

(57) ABSTRACT

An artificial insemination system and its use requires a sheath, a pumping device, and an AI gun-straw of semen assembly. As intended for the present invention, the AI gun-straw of semen assembly is insertable into the central lumen of the sheath. There, the distal end of the straw of semen is insertable into a slidable insert, which, in turn, is insertable into a transfer tunnel. Last, the transfer tunnel opens into an ejection port in the ventrodistal portion of the sheath.

As further intended for the present invention, the pumping device is attachable to the proximal portion of the sheath for inflating a balloon on the distal portion of the sheath. The balloon, when expanded within the uterine body, anchors the ejection port within the uterine body during insemination.

### RELATED APPLICATIONS

The present application is related to U.S. Pat. No. 7,056,279 B2, issued Jun. 6, 2006, for DEVICE AND METHOD FOR ARTIFICIAL INSEMINATION OF BOVINES AND OTHER ANIMALS, by Steven Verberckmoes, Ann Van Soom, Aart De Kruif, included by reference herein.

The present application is related to U.S. Pat. No. 4,173,227, issued Nov. 6, 1979, for INJECTING GUN FOR ANIMALS, IN PARTICULAR FOR THE ARTIFICIAL INSEMINATION OF CATTLE, by Robert E. J. Cassou, Bertrand M. E. Cassou, included by reference herein.

The present application is related to U.S. Pat. No. 6,071,231, issued Jun. 6, 2000, for DEVICE AND METHOD FOR ARTIFICIAL INSEMINATION OF BOVINES AND OTHER ANIMALS, by Marco Antonio Hidalgo Mendoza, William A. Niven, included by reference herein.

### FIELD OF THE INVENTION

The present invention generally relates to an artificial insemination system for bovines, and to the method of use thereof for depositing semen in the uterine body.

### BACKGROUND OF THE INVENTION

Artificial insemination (AI) is widely used by the dairy and beef industries to breed cattle without the necessity of maintaining bulls on the premises. Importantly, AI allows producers to make swifter genetic gains in their animals because of the availability of semen from genetically superior bulls. The origin of AI in cattle dates to 1899, when Ivanoff of Russia first successfully inseminated cattle artificially. Artificial insemination was subsequently practiced in Russia by 1931, in Denmark by 1936, and then in the United States by 1938. Today, a large percentage of cattle worldwide are bred artificially.

In the early years of AI, inseminators bred cows by depositing semen in the animal's posterior cervix (using a pipette). By the 1940's, the aforementioned pipette was replaced by the insemination rod, a device which allowed inseminators to deposit semen in the uterus, rather than in the posterior cervix. This insemination rod consists of three parts: an AI gun, a straw of semen, and a flexible protective sheath; in operation, the inseminator inserts the straw of semen into the AI gun, and then inserts the gun-straw assembly into the protective sheath. During the process of insemination, the inseminator guides the semen loaded AI rod through the cervix and into the uterus, whereupon the semen is ejected. The technique of insemination using an AI rod is still in use today. An example of an AI rod used to inseminate cattle is disclosed in U.S. Pat. No. 4,173,227.

The likelihood of conception due to artificial insemination varies with the site of semen deposition within the reproductive tract, as determined in a review by F. Lo'pez-Gatius in Theriogenology, 2000, vol. 53, pp. 1407-1414. Data in the review shows that conception rates are higher following insemination of semen in the uterine horns compared to deposition of semen farther caudal in the reproductive tract, e.g., in the uterine body, cervix, or vagina. Consequently, the preferred site for deposition of semen is the uterine horns; however, because uterine horn insemination is technically difficult, most inseminators are taught to deposit semen in the easier-to-reach uterine body.

Of concern, however, is the ability of inseminators to consistently deposit semen in the aforementioned uterine body. A study published by Peters et al. in J. Anim. Sci., 1984, vol. 59, pp. 1671-1683, shows that inseminators vary in their ability to deposit semen in the uterine body. In their study, inseminators were asked to place an AI rod tip in the uterine body of an excised reproductive tract and deposit a dye. Surprisingly, only 39 percent of rod tips were accurately placed in the uterine body. Other rod tip placements were as follows: cervix, 25 percent, right uterine horn, 23 percent, and left uterine horn, 13 percent. Based on these results, the authors of Penn State Spec. Circ. 314 stated, in part, “ . . . that accurate artificial insemination requires the ability to identify the target area [uterine body] and proper positioning of the insemination rod . . . . It further demonstrates the need for routine retraining and updating of both professional and owner-inseminators.”

Since the study by Peters et al. (1984), and others, several devices have been invented in an attempt to improve the technique of artificial insemination in cattle. In U.S. Pat. No. 7,056,279 B2, Verberckmoes et al. describe an AI rod that allows the inseminator to more easily advance the rod through the uterine body and into the uterine horns (the site described by F. Lo'pez-Gatius {2000} as the optimal site for semen deposition). The device consists of 3 sliding cylinders, the last of which is flexible, permitting the tip of the AI rod to bend with the curvature of the uterine horns, making uterine horn insemination easier.

Another device, invented by Mendoza et al. and disclosed in U.S. Pat. No. 6,071,231, involves flushing semen from the cervix into the uterus with a diluent. The invention requires that the inseminator perform the following steps: first, position the tip of a pipette into the cervix (with the aid of a vaginal speculum); second, inflate a balloon on the pipette tip (to seal the space between the tip and the cervix); and third, use a syringe filled with diluent to flush semen from the straw, through the cervix, and into the uterus.

Interestingly, neither of the aforementioned devices nor methods have been adopted by the AI industry. Rather, the industry has opted to find devices and methods that improve insemination in the uterine body.

It is therefore an object of the present invention to provide a device and method for artificial insemination of bovines that increases the accuracy of rod tip placement in the uterine body.

It is a further object of the present invention to provide a device and method for artificial insemination of bovines that decreases the incidence of rod tip placement in the cervix or uterine horns during uterine body insemination.

It is a further object of the present invention to provide a device and method for artificial insemination of bovines that minimizes the amount of training and retraining required to perform artificial insemination.

It is a further object of the present invention to provide a device and method for artificial insemination of bovines that maximizes conception rates following uterine body insemination.

It is a further object of the present invention to provide a device and method for artificial insemination of bovines that is easy to use, disposable, and cost effective.

These and other objects of the present invention will be apparent in the following description.

### SUMMARY OF THE INVENTION

A system for artificially inseminating bovines essentially includes a sheath, a pumping device, and an AI gun-straw of semen assembly. More specifically, the AI gun-straw of semen assembly is insertable into the central lumen of the sheath. There, the distal end of the straw of semen is insertable into a slidable insert, which, in turn, is insertable into a transfer tunnel. As intended, the distal end of the transfer tunnel opens into an ejection port in the ventrodistal portion of the sheath.

Further, the pumping device is attachable to an air port on the proximal portion of the sheath, and is usable there for inflating a balloon on the distal portion of the sheath. The balloon, when expanded within the uterine body, anchors the ejection port within the uterine body during insemination.

It is intended that the sheath for the present invention is disposable and may be discarded after use.

When using the system, first, the AI gun-straw of semen assembly is inserted into the long, central lumen of the sheath, the distal end of the straw of semen is press fit into the insert, and the entire assembly, AI gun-straw of semen-insert, is slid axially until the insert engages the transfer tunnel in the second part of the sheath. Next, the pumping device is attached to the air port. The inseminator then guides the assembled invention into the vagina, through the cervix, and into the uterine body; once in the uterine body, the inseminator inflates the balloon using the pumping device, pulls the AI rod caudally until the balloon makes firm contact with the cervix, and, finally, ejects the semen.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective view of the artificial insemination system positioned within the reproductive tract of a cow with the balloon inflated inside the uterine body;

FIG. 2 is a plan view of the major components of the artificial insemination system;

FIG. 3 is a longitudinal sectional view of the sheath and insert in combination;

FIG. 4 is an enlarged, side sectional view of the insert;

FIG. 5 is an enlarged, end sectional view of the distal portion of the sheath in the area of the balloon and ejection port;

FIG. 6 is an enlarged, end sectional view of the sheath in the area of the air line;

FIG. 7 is a partial sectional view of the pumping device;

FIG. 8 is a longitudinal sectional view of the sheath, straw of semen, and artificial insemination gun with connecting lines to show their respective cooperation;

FIG. 9 is a sectional view of the assembled invention, including a partial sectional view of the pumping device;

FIG. 10 is a sectional view of the insert and distal portion of the sheath, each shown separately and in combination, in a modified embodiment of the present invention;

FIG. 11 is a sectional view of the insert and distal portion of the sheath, each shown separately and in combination, in a further modified embodiment of the present invention;

FIG. 12 is a partial sectional view of the proximal portion of the sheath in combination with a plan view of the air port and quick-connect type connector in a modified embodiment of the present invention;

FIG. 13 is a partial sectional view of the proximal portion of the sheath in combination with a plan view of the air port and quick-connect type connector in a further modified embodiment of the present invention; and

FIG. 14 is a partial sectional view of the proximal portion of the sheath in combination with a plan view of the air port and quick-connect type connector in a further modified embodiment of the present invention.

For purposes of clarity and brevity, like elements and components will bear the same designations and numbering throughout the Figures.

### DESCRIPTION OF THE PREFERRED EMBODIMENT

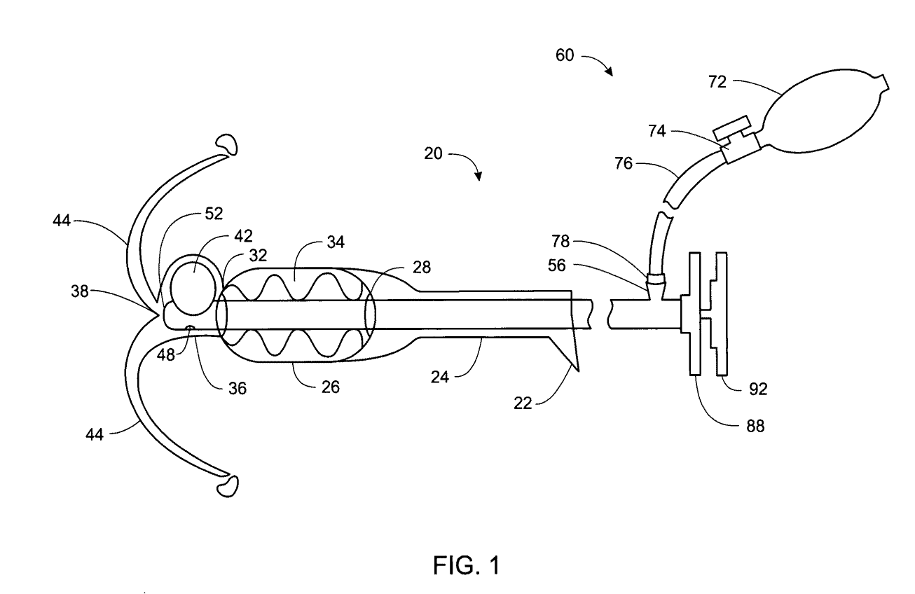

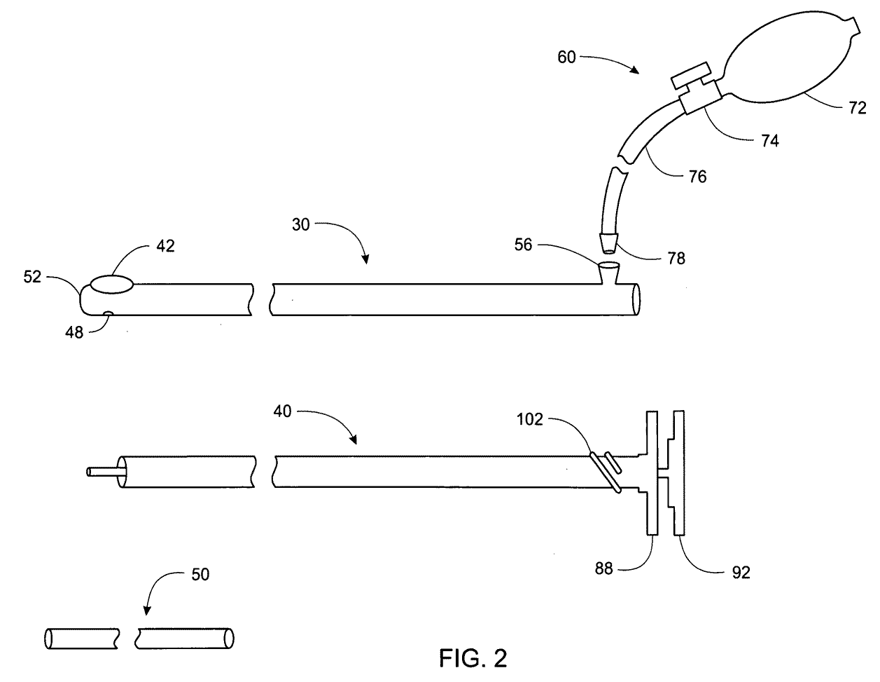

The present invention is a system and method for artificial insemination of bovines. The system of the present invention is shown in FIG. 1, and is generally designated as 20. In accordance with its intended use, the system 20 is shown inserted within the reproductive tract of the cow. With reference to the cow's anatomy, system 20 extends from outside the cow, through the vagina 24 and cervix 26, and into the uterine body 36, ending near the bifurcation 38 of the uterine horns 44. In further detail, the vagina 24 is shown as a cavity or potential space between the cow's vulva 22 and cervix 26. At its anterior end, the vagina 24 forms a blind pouch that encircles the posterior end of the cervix 26 and its opening, the posterior cervical os 28. The body of the cervix 26, or cervix proper, which consists of muscle and dense connective tissue, is shown with three or four annular rings or folds 34 that project into its canal; last, the anterior opening of the cervix, the anterior cervical os 32, projects into the uterine body 36—an area that averages just ⅝ths of an inch in length (ranging between ⅜ths and ⅞ths of an inch in length) as described in Penn State Spec. Circ. 314. Major components of the system 20 may be appreciated by referring initially to FIG. 2.

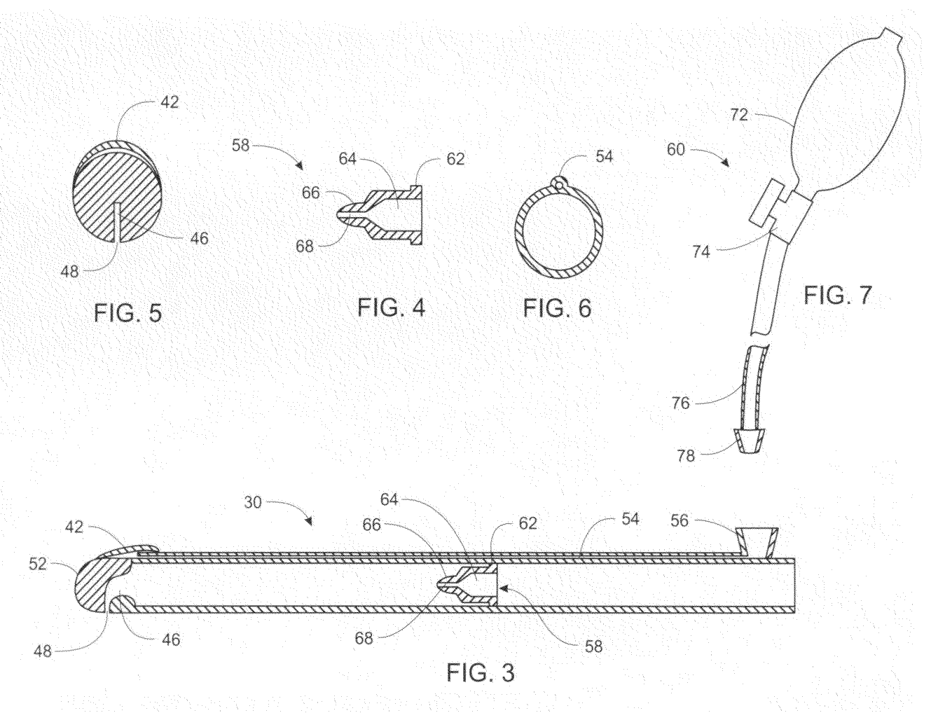

As shown in FIG. 2, major components of the artificial insemination system include a sheath 30 (with an air port 56 on the proximal portion of the sheath 30 and a balloon 42 on its distal portion), an AI gun 40, a straw of semen 50, and a pumping device 60. Structural details of each component and the assembly of the system 20 may be appreciated in FIGS. 3-8.

As depicted in FIG. 3, the sheath 30 is a flexible cylinder and protective structure that defines the outer surface of the AI rod. More specifically, the sheath 30 is comprised of a first part—a long, thin-walled tube with a large, central lumen—that houses the AI gun 40 and straw 50 of semen, and of a second part that consists of a balloon 42, a transfer tunnel 46, and an ejection port 48. The sheath 30 ends as the sheath tip 52, a short, solid, and conical structure that helps Minimize injury to the reproductive structures during insemination.

The balloon 42 is connected to a small diameter lumen that spans most of the top length of the sheath 30; the lumen, an air line 54 (also shown in FIG. 6) connects the balloon 42 to the air port 56. Conveniently, the air port 56 projects from the proximal portion of the sheath 30 as a short, hollow tube for connection to the pumping device 60. Consequently, the air port 56 and air line 54 define a flow path for air between the balloon 42 and the pumping device 60. The air port 56 and air line 54 may be molded with the sheath 30 during its manufacture (usually from polyvinyl chloride). Alternatively, the air line 54 and air port 56 may be attached onto the sheath 30 in conjunction with the balloon 42. The balloon 42, typically made of rubber or silicone, is attached onto the dorsodistal aspect of the sheath 30. Importantly, other flowable substances, such as a gas or a fluid, may be substituted for air in the present or modified embodiments of the artificial insemination system 20. Similarly, other inflating devices that provide the desired function may be substituted for the pumping device 60 described herein.

The transfer tunnel 46, present in the second part of the sheath 30, is a narrow, tubular passageway comprised of proximal and distal end openings. The proximal end opening, frusto-conical in shape, provides a receptacle for the slidable insert 58 (shown in the middle part of FIG. 3). The distal end opening, the ejection port 48, provides a means of directing semen out the ventrodistal aspect of the sheath 30.

Components of the slidable insert 58 may be appreciated better in the enlarged, sectional view of FIG. 4. In accordance with the invention, the insert 58 is comprised of three components: a proximal flange 62, an insert body 64, and a distal extension 66. The proximal flange 62 extends from the insert body 64 at a right angle, and has an outer diameter corresponding to the inner diameter of the sheath 30; the flange 62 helps guide and stabilize the insert 58 as it slides axially within the sheath 30. The insert body 64 is hollow and slightly frusto-conical in shape, and provides a receptacle for the distal end of the straw 50 of semen. Finally, the distal extension 66 tapers slightly into a conical shape, and engages the transfer tunnel 46 in the second part of the sheath 30. Conveniently, a small, central canal 68 within the insert extension 66 provides passage therethrough for semen from the straw 50 to the transfer tunnel 46 and, ultimately, to the ejection port 48.

As illustrated in FIG. 5, the ejection port 48 and balloon 42 are located on opposite sides of the sheath 30. In the deflated state, the balloon 42 fits closely to the contour of the cylindrical sheath 30 to maintain the smallest possible outer diameter of the sheath 30; more specifically, the outer diameter of the sheath 30, and attached structures, must be small enough to pass through the narrow canal and protruding folds 34 of the cervix 26, as shown in FIG. 1. Once inside the uterine body 36, the balloon 42 is expanded to prevent slippage of the ejection port 48 back into the cervix 26.

The pumping device 60, which attaches to the proximal portion of the sheath 30, provides a means for expanding the balloon 42 present on the distal portion of the sheath 30. As illustrated in FIG. 7, the pumping device 60 is comprised of a compressible bladder 72, a pressure relief valve 74, a substantially flexible tube 76, and a quick-connect type connector 78. The compressible bladder 72 and pressure relief valve 74 are of a design used in other inventions, such as the manually operated blood pressure cuff. The quick-connect type connector 78 is formed as a short, hollow cylinder with a slight taper or conical shape that press fits into the air port 56. Accordingly, the pumping device 60 of the present invention provides a practical method for expanding the balloon 42 while inseminating cattle in a variety of environments.

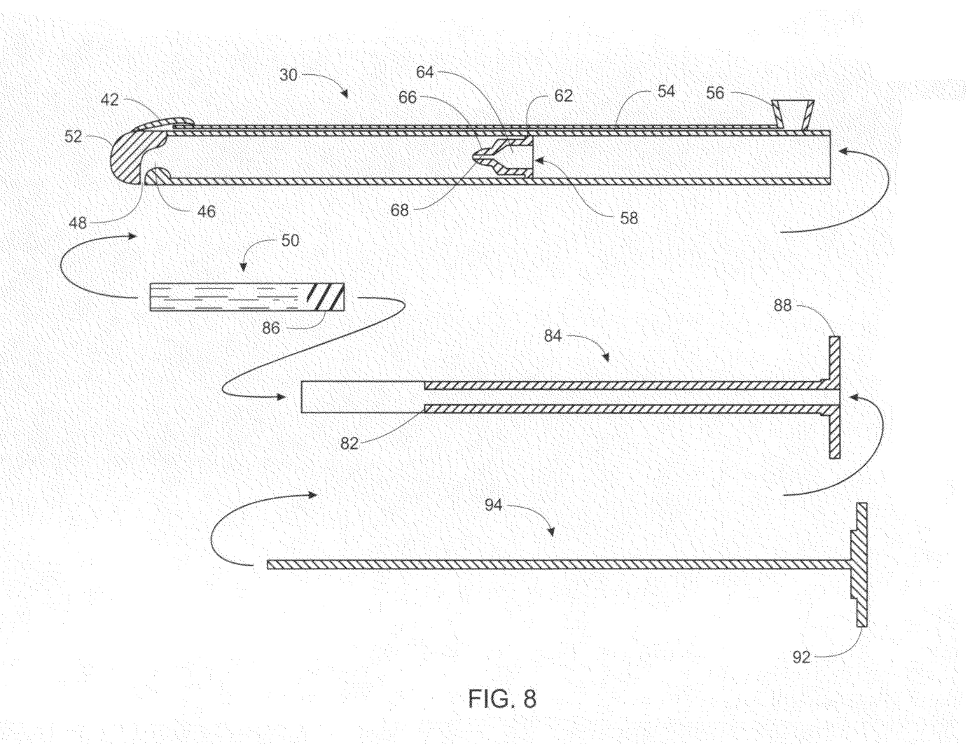

As intended for the present invention, the AI gun 40 (and straw 50) is insertable into the sheath 30. As shown in FIG. 8, the AI gun 40 is comprised of a push rod 94 and a long, tubular body 84 with an axial passageway. The tubular body 84 of the AI gun 40 consists of a first part that provides an axial passage therethrough for the push rod 94, and of a second part that fixes the straw 50 against an inner shoulder 82. In operation, the tip of the push rod 94 slides past the inner shoulder 82 of the tubular body 84 to contact the closing element 86 of the straw 50 to evacuate the semen. The length of the push rod 94 slightly exceeds that of the tubular body 84 to ensure full evacuation of semen from the straw 50 by the closing element 86.

An annular handling disc 88 that provides for easy manipulation of the AI gun 40 forms the head or most proximal part of the tubular body 84. Similarly, a head or annular handling flange 92 forms the most proximal part of the push rod 94. The push rod 94 and tubular body 84 are typically maintained as a unit, even though the slidably mounted push rod 94 can be completely withdrawn from the tubular body 84.

Referring to FIG. 2, the outside surface of the proximal part of the tubular body 84 projects coiled ridges 102, which form a helical profile that fix thereon the sheath 30 to the tubular body 84. Other methods of fixing the sheath 30 to the tubular body 84, including the use of a slidable retaining ring, may be used with the system 20. In general, an AI gun 40 of any type that can be fixed within the central lumen of the sheath 30 may be used in the operation of the system 20. The AI gun 40 is most commonly constructed of stainless steel.

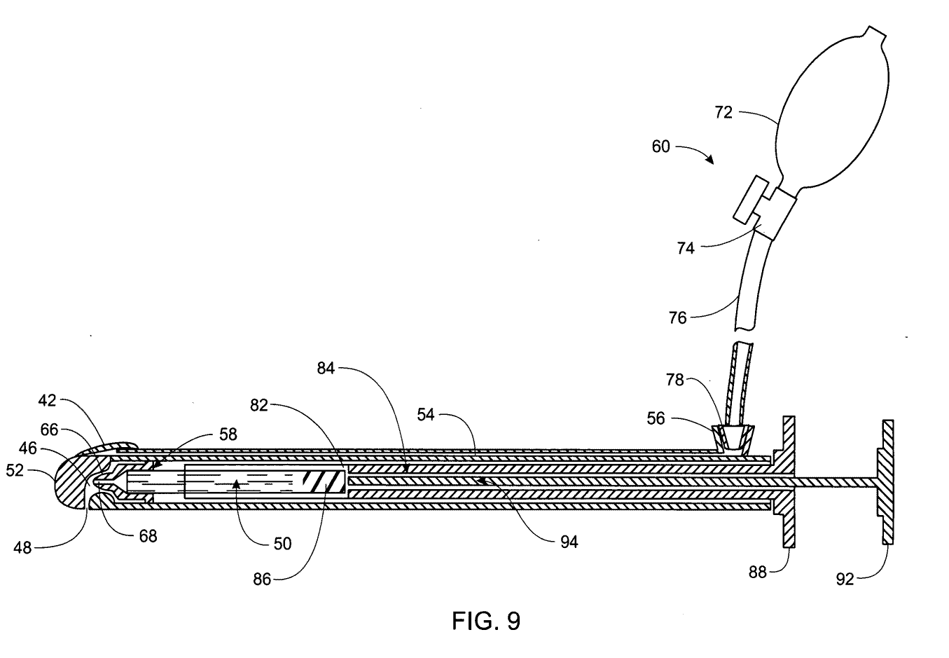

A depiction of the assembled invention—the sheath 30, AI gun 40, straw 50, and pumping device 60—is shown FIG. 9: first, the tip of the push rod 94 is shown inserted next to the closing element 86 in the proximal part of the straw 50; second, the distal end of the straw 50 is shown inside the insert body 64; and third, the distal extension 66 is shown engaged with the transfer tunnel 46 in the second part of the sheath 30. Also in FIG. 9, the proximal end of the sheath 30 is abutted against the handling disc 88 of the tubular body 84; the push rod 94 is extended in the ready position; and the pumping device 60 is connected to the air port 56.

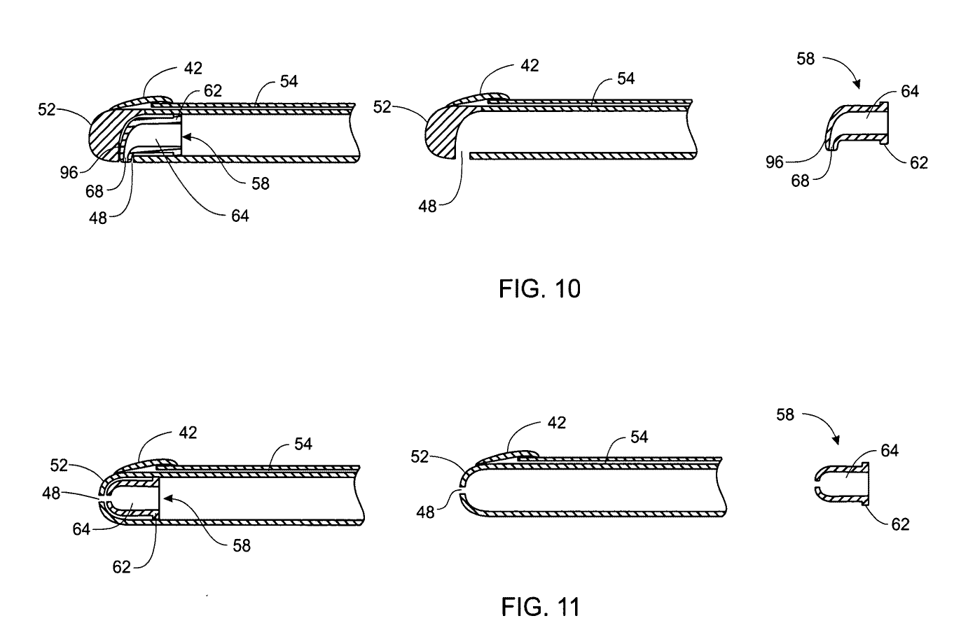

FIG. 10 is an illustration of the insert 58 modified to include a ventral, rather than distal, extension. Conveniently, the ventral extension 96 snaps directly into the ejection port 48 of the sheath 30, thereby obviating the need for a transfer tunnel 46 within the sheath 30.

In a further modification, the insert 58 is shown in FIG. 11 without a distal or ventral extension; as intended, the modified insert 58 directly abuts an ejection port 48 in the distal end of the sheath 30. Additionally, the distal portion of the sheath 30 is hollow rather than solid as in the previous sheath 30.

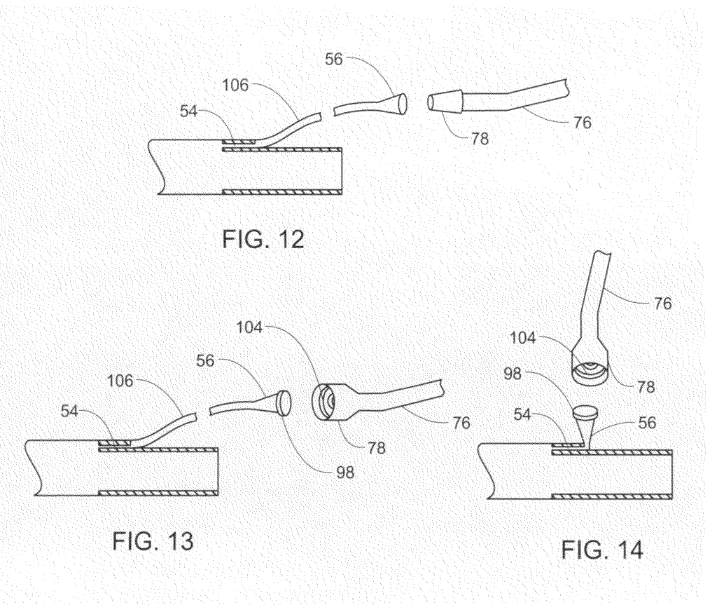

A modified embodiment of the air line 54 and proximal portion of the sheath 30 is shown in FIG. 12. In this embodiment, the air line 54 is extended beyond the proximal end of the sheath 30 by a small diameter, flexible tube 106; concurrently, the air port 56 is relocated from the proximal portion of the sheath 30 to the end of the flexible tube 106.

Finally, FIGS. 13 and 14 illustrate modified embodiments of the elements that provide for attachment of the sheath 30 to the pumping device 60. In FIG. 13, the air port 56 is shown as a short, hollow cylinder with a proximal ridge 98; whereas the quick-connect type connector 78 is shown as an outer tubular shell with internal threads 104. In operation, the quick-connect type connector 78 is screwed, rather than press fit, into the air port 56.

In FIG. 14, the air port 56 is shown as a short, hollow cylinder with a proximal ridge 98, same as shown in FIG. 13; whereas the quick-connect type connector 78 is shown as an outer tubular shell with internal threads 104, also shown in FIG. 13. In operation, the quick-connect type connector 78 is screwed into the air port 56 present on the proximal portion of the sheath 30.

In accordance with a preferred embodiment of the invention, the procedure of artificial insemination of a cow by employing the device of FIG. 9 will now be described hereinbelow.

A straw 50 of semen that holds either 0.25 mL or 0.50 mL of fresh or, more commonly, frozen-thawed semen is prepared for insemination according to the instructions provided by the semen supplier. As a matter of routine, the user opens the distal end of the straw 50 with a straw cutter before inserting the proximal end of the straw 50 into the distal end of the tubular body 84. The straw 50 is then slid axially therein until the proximal end of the straw 50 abuts the inner shoulder 82 of the tubular body 84. As shown in FIG. 9, the distal end of the loaded straw 50 extends beyond the tubular body 84.

Next, the user inserts the AI gun-straw assembly into the open end of the sheath 30 until the distal end of the straw 50 contacts the insert 58. Using one hand, the user press fits the straw 50 into the insert body 64 by pushing the AI gun 40, while using the other hand to lightly squeeze the insert 58 through the sheath 30. Finally, the AI gun-straw-insert assembly is slid axially until the distal extension 66 engages the transfer tunnel 46 in the second part of the sheath 30; concurrently, the first part of the sheath 30 fixes to the tubular body 84 (via the coiled ridges 102 shown in FIG. 2).

After inserting the AI gun-straw assembly into the sheath 30, the user attaches the pumping device 60 to the sheath 30 by press fitting the quick-connect type connector 78 into the air port 56. The attachment between the air port 56 and the quick-connect type connector 78 is maintained by friction. With the artificial insemination system 20 assembled, the user is ready to inseminate the cow. The insemination procedure may be appreciated best by further reference to the cow's reproductive anatomy in FIG. 1.

The inseminator places one hand in the rectum of the cow, while the other hand is used to insert the AI rod into the cow's vagina 24. Since the walls of the vagina 24 consist of thin layered muscle and loose connective tissue, the tip of the AI rod can be easily felt by the inseminator's hand in the rectum. After the AI rod is about 6 inches inside the vagina 24, the inseminator raises the rear of the AI rod to a somewhat level position and slides it forward toward the cervix 26. Next, the inseminator grasps and gently pushes the cervix 26 forward to straighten the vagina 24, thereby allowing the AI rod to pass freely into the posterior cervical os 28; then, by bending and twisting the cervix 26, the inseminator threads the cervix 26, with its cervical folds 34, over the AI rod. The exact location of the AI rod within the hard and firm cervix 26 is difficult to determine; however, as the tip of the AI rod enters the soft and spongy uterus, the inseminator is once again able to feel the exact position of the AI rod.

To anchor the tip of the AI rod in the uterine body 36, the inseminator rotates the air port 56 to a dorsal position and squeezes the compressible bladder 72 to expand the balloon 42 on the distal portion of the sheath 30; air within the balloon 42 is maintained by the pressure relief valve 74 on the pumping device 60. The inseminator then pulls the AI rod caudally until the balloon 42 makes firm contact with the cervix 26 which positions the ejection port 48 within the uterine body 36. Finally, the inseminator shifts the push rod 94 forward, causing the closing element 86 in the straw 50 to move toward the ejection port 48, forcing semen out of the sheath 30. The straw 50 is empty when the annular handling flange 92 of the push rod 94 contacts the annular handling disc 88 of the tubular body 84. With insemination completed, the inseminator opens the pressure relief valve 74 on the pumping device 60 to deflate the balloon 42, and removes the system 20 from the cow.

The pumping device 60 is detached from the sheath 30 by pulling the quick-connect type connector 78 out of the air port 56. Then, the AI gun 40 is withdrawn from the central lumen of the sheath 30 by gripping the sheath 30 with one hand while pulling outwardly on the AI gun 40 with the other hand. Finally, the empty straw 50 is withdrawn from the tubular body 84. Both the sheath 30 and empty straw 50 are disposed,

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. An apparatus for the artificial insemination of bovines, which comprises:
a sheath for enclosing within an insertable AI gun-semen filled tube assembly;
a means, for attaching said sheath to an inflation device, connected to the proximal portion of said sheath;
a means, for anchoring the distal portion of said sheath in the uterine body, adhesively connected to the dorsodistal portion of said sheath; and
a means, for providing an air conduit between components on the dorsal aspect of said sheath, fixedly connected to said means for anchoring the distal portion of said sheath in the uterine body, and connected to said means for attaching said sheath to an inflation device.

2. The apparatus of claim 1 wherein said means for attaching said sheath to an inflation device comprises an air port.

3. The apparatus of claim 1 wherein said means for anchoring the distal portion of said sheath in the uterine body comprises an expandable balloon.

4. The apparatus of claim 1 wherein said means for providing an air conduit between components on the dorsal aspect of said sheath comprises an air line.

5. An apparatus for the artificial insemination of bovines, which comprises:
a sheath for enclosing within an insertable AI gun-semen filled tube assembly;
an air port, for attaching said sheath to an inflation device, connected to the proximal portion of said sheath;
an expandable balloon, for anchoring the distal portion of said sheath in the uterine body, adhesively connected to the dorsodistal portion of said sheath; and
an air line, for providing an air conduit between components on the dorsal aspect of said sheath, fixedly connected to said expandable balloon for anchoring the distal portion of said sheath in the uterine body, and connected to said air port for attaching said sheath to an inflation device.

6. The apparatus of claim 5, wherein said semen-filled tube is a straw of semen.

7. The apparatus of claim 5, wherein said air port includes a short, hollow tube.

8. An apparatus for the artificial insemination of bovines, which comprises:
a sheath for enclosing within an insertable AI gun-straw of semen assembly;
an air port, having a short, hollow tube, for attaching said sheath to an inflation device, connected to the proximal portion of said sheath;
an expandable balloon, for anchoring the distal portion of said sheath in the uterine body, adhesively connected to the dorsodistal portion of said sheath; and
an air line, for providing an air conduit between components on the dorsal aspect of said sheath, fixedly connected to said expandable balloon for anchoring the distal portion of said sheath in the uterine body, and connected to said air port for attaching said sheath to an inflation device.

9. The apparatus of claim 8, further comprising:
a slidable insert having a central hollow body for receiving the open end of said straw of semen, and having a distal extension for insertion of the insert into the second part of the sheath, said insert slidably connected to the central, elongate lumen of said sheath.

10. The apparatus of claim 8, further comprising:
a transfer tunnel having a proximal end opening for receiving said insert in the second part of said sheath, and having a distal end opening, an ejection port, for directing semen out of said sheath.

11. The apparatus of claim 8, further comprising:
a quick-connect type connector, for attaching an inflation device to said air port in a quick manner, fixedly attached to the inflation device.

12. The apparatus of claim 8, further comprising:
a pumping device, having a compressible bladder, for expanding said balloon, operatively connected to said air port for attaching said sheath to an inflation device.

13. A method for artificial insemination of bovines, said method involving the use of a sheath with a balloon, an air port, and an attachable inflation device, and involving the use of an AI gun-straw of semen assembly that is insertable into the central, elongate lumen of said sheath, said method comprising the steps of:
inserting said AI gun-straw of semen assembly into the lumen of said sheath;
attaching the inflation device to said air port on the proximal portion of said sheath;
inserting the AI gun-straw of semen-sheath assembly through the cervix and into the uterine body;
expanding said balloon on the distal portion of said sheath using the inflation device;
pulling said AI gun-straw of semen-sheath assembly caudally until the expanded balloon makes firm contact with the cervix;
ejecting semen from said sheath via an ejection port; and
removing said AI gun-straw of semen-sheath assembly from the reproductive tract of the cow.

14. The method of claim 13, further comprising press fitting the open end of said straw of semen into a slidable insert within the central lumen of said sheath;

15. The method of claim 13, further comprising press fitting the distal extension of said slidable insert into the proximal end opening of a transfer tunnel in the second part of said sheath.
